(19) United States
(12) Patent Application Publication
(10) Pub. No.: US 2010/0056957 A1
(43) Pub. Date: Mar. 4, 2010

(54) METHOD AND DEVICE FOR THE REHABILITATION AND/OR CORRECTION OF POSTURAL SYMMETRY IN STATIC OR DYNAMIC SITUATIONS

(75) Inventors: Nicolas Vuillerme, Francin (FR); Yohan Payan, Allevard (FR); Jaques Demongeot, Sassenage (FR)

Correspondence Address: Howard IP Law Group, P.O. Box 226, Fort Washington, PA, 19034, US

(73) Assignees: UNIVERSITE JOSEPH FOURIER, Grenoble Cedex 9 (FR); CENTRE NATIONAL DE LA RECHERCHE SCIENTIFIQUE, Paris Cedex 16 (FR)

(21) Appl. No.: 12/304,057

(22) PCT Filed: Jun. 11, 2007
(86) PCT No.: PCT/FR07/51409

§ 371 Date: Mar. 18, 2009

(30) Foreign Application Priority Data
Jun. 9, 2006 (FR) 0652084

(51) Int. Cl. A61B 5/103

(52) U.S. Cl. 600/587

(57) ABSTRACT

A method for rehabilitation or correction of the postural symmetry of a person in static or dynamic situations includes the following steps: monitoring at least one parameter of at least one body part; detecting whether the parameter and/or a combination of parameters falls short of, or exceeds, a threshold value; and if so, warning the person by means of a lingual electrostimulation signal and informing the person of a postural response to be carried out in order to bring the value back to the threshold value.

### FIELD OF THE INVENTION

The present invention generally relates to a method for correcting a static or dynamic posture. More specifically, the present invention relates to the correction or the rehabilitation of an otherwise-stable posture to increase its postural symmetry.

### DISCUSSION OF PRIOR ART

In many activities, the control of the body balance is wrongly considered as the sole crucial factor.

Thus, in sports, a significant element for optimizing performances is not as much the acquisition of a stable balance position as, above all, in the optimization of a non-traumatic positioning of various body segments, among which the arms, the forearms, the hand, the trunk, the legs . . . An optimization is currently obtained by trainings during which the posture is most often corrected by another person than the athlete, based on statistical data. This correction, which also takes into account the reactions of the athlete's organism however takes time and implies a long training. Further, this correction can only be slowly individualized and does not guarantee the absence of lesions.

This problem is further enhanced after a trauma such as a tendon rupture, a muscle tearing, a spraining or fracture of a joint or of a bone, when after a long time for which he or she is not allowed to move, the athlete must recover an optimal position which may be different from his optimal position before the trauma. Further, the athlete must additionally cope with a most often unconscious psychological unwillingness to use the body segment.

Generally, any person, athletic or not, may, after a trauma, have to follow a rehabilitation aiming either at correcting a position which has caused the trauma or learn to control his body again after a particularly severe trauma such as an amputation.

Currently, such a rehabilitation or position correction aims at ensuring the stability and the balance of the individual to prevent him from falling. They do not take into account the high probability of occurrence of lesions sometimes outside the trauma area.

Apart from a post-traumatic context, as has been previously mentioned in the case of an athlete, a bad positioning of body segments may cause lesions in the practice of many professional activities.

For example, surgeons are led during interventions to take stable positions that they must often hold for time periods which may be long. The stabilizing of their postures occurs naturally or is acquired by experience. However, a stable balance position may cause traumas, especially on the vertebral column or the upper limbs, given the motions constraints above an operation area which may be difficult to get to.

Another category of people likely to incur lesions although they have acquired a stable balance position are disabled people using a wheelchair. They must find a specific stable position in their chair. Such a position may be found quite rapidly. However, it can frequently be observed that such a position, even recommended by health staff, causes deformations of the vertebral column.

Similarly, people affected by pathologies of the skeleton, especially of the vertebral column, such as scoliosis, lumbar pain, or neck pain, unconsciously tend to adopt a posture which prevents the compensation of their pathology, or even enhances the pathology. Frequently, the unconscious position taken by such people is the cause of other pathologies, especially the forming of plantar ulcers.

Other people likely to require a posture correction are individuals, for example, older people, having a sensory perception impairment which affects their sense of balance. To dispense these people from using a walking frame, while enabling them to remain at home, fall detection systems have already been provided. However, such systems only enable to detect whether a person has fallen or risks falling. These systems do not enable to detect the occurrence or the risk of occurrence of lesions due to a bad position, although this position may be perfectly stable and exhibit no risk of falling for the person. Further, such a bad position may seem optimal to the person as well as to the people around him—including the health staff—to enable him to carry out daily tasks.

Currently, any rehabilitation or posture correction imposes using bulky specialized equipment aiming at placing the body segments in a statistically optimal position. In case of a divergence, the equipment replaces the segments in their position or warns an attendant of the need to intervene to correct the position. This is particularly costly, in terms of human intervention as well as in terms of means. Further, the necessary equipment and personnel can often only be found in large agglomerations, most often within hospital centers. This creates constraints which are often impossible to manage for a working person who only requires a posture correction to avoid lesions due to a bad position. Such a person frequently renounces to the correction because of the constraints. A person reaching the end of a post-trauma rehabilitation may be confronted to similar difficulties. Such material constraints are enhanced by the lack of material and of personnel of hospitals which may have difficulties to integrate in their structures people who are not or no longer relevant to their care activity.

For example, a rehabilitation enables a person having a vertebral column affection to learn to move or to perform daily life actions without falling. However, such rehabilitations only concern the balance control without at all taking into account the possible lesions caused by the acquired position, which is only optimized to avoid a fall.

In practice, whatever the aimed activity or the circumstances, posture correction procedures and rehabilitation systems are limited either to enable the acquisition of a stable posture in which an activity can be performed with no risk of falling, or to the learning to use a damaged or prosthetic limb. However, none of these systems takes into account lesions which would be likely to appear on other parts of the body due to the adopted balance posture, which balance posture is often recommended to avoid falls.

For example, after the fitting of a lower limb prosthesis, the patient learns to progressively let his weight bear on the prosthesis until he has recovered a stable gait. However, instinctively, once the rehabilitation period is over, the bearer of a prosthesis will unconsciously or consciously tend to limit the use of the equipped side and will let its weight and efforts bear more on the side which is not fitted with the prosthesis, which will in the end cause irreversible lesions of the healthy limb. This additional traumatic effort will however be undetectable by the environment, even health professionals, especially due to the absence of falls.

### SUMMARY OF THE INVENTION

The present invention aims at providing a device of rehabilitation and of correction of a posture to prevent the occurrence of lesions. More specifically, the present invention aims at providing a device of rehabilitation and of correction of the postural symmetry in dynamic or static situations.

The present invention aims at providing such a device which is simple and likely to be used by a person alone with no supervision and no installation.

The present invention also aims at providing such a device which adapts to the physical characteristics, to the sensorimotor capacities, and to the needs of the person.

The present invention also aims at providing such a device which is usable in a static position as well as in a dynamic position.

To achieve these and other objects, the present invention provides a method of rehabilitation or of correction of the postural symmetry of a person in static or dynamic situations, comprising the steps of monitoring at least one parameter of at least one body segment; detecting whether the parameter and/or a combination of parameters has passed a high or low threshold value; and warning the person by means of an electrical tongue stimulation signal that the threshold value has been passed.

According to an embodiment of the present invention, the coding of the electrical tongue stimulation signal is performed according to the location of the passing area.

According to an embodiment of the present invention, the coding of the electrical tongue stimulation signal is performed according to the amount of passing.

According to an embodiment of the present invention, the coding of the electrical tongue stimulation signal is performed according to instructions for the displacement of at least one body segment to compensate for the passing.

According to an embodiment of the present invention, the method further comprises after the detection of a passing, the triggering of at least one actuator capable of causing a compensation of the passing.

According to an embodiment of the present invention, the actuator is an electromyographic actuator or a sensory stimulator capable, for example, of applying a tactile, tendinous, or muscular vibration.

The present invention also provides a device of rehabilitation or correction of the postural symmetry in static or dynamic situations of a person, comprising at least one sensor placed on at least one body segment of the person; and a processing unit capable of detecting whether the parameter provided by the sensor(s) and/or a combination of parameters have passed at least one given threshold and capable of sending a signal representative of the passing to at least one actuator.

According to an embodiment of the present invention, an actuator is a device of electrical stimulation of the tongue.

According to an embodiment of the present invention, an actuator is an actuator capable of causing a postural reaction, such as an electromyographic actuator or a sensory stimulator.

### BRIEF DESCRIPTION OF THE DRAWINGS

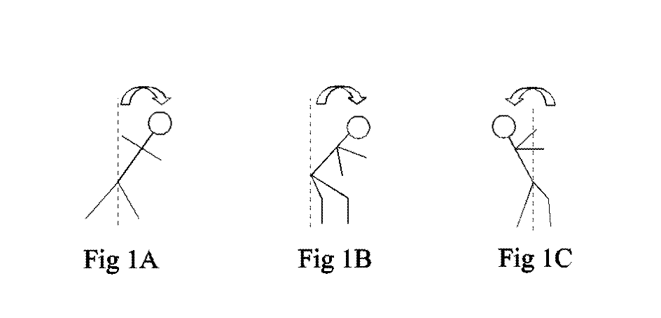

The foregoing objects, features, and advantages of the present invention, as well as others, will be discussed in detail in the following non-limiting description of specific embodiments in connection with FIGS. 1A, 1B, and 1C which illustrate a person in motion.

### DETAILED DESCRIPTION

Generally, the inventors have shown that all lesions not directly linked to an existing pathology or in the absence of a pathology result from bad positioning of body segments, which break the natural postural symmetry.

Indeed, when the human body is in a stable balance posture, it naturally exhibits a postural symmetry. For example, the upper limbs balance with one another to maintain the bary-center of the body at a balance position. After a trauma, or in the course of a professional activity or of a sport, this symmetry balancing is broken. The body finds a stable balance position, but to the detriment of some of its segments which are then damaged.

Rehabilitation devices and methods developed up to now only aim at the recovery and the relearning of a balanced posture without taking into account the degree of symmetry, or rather of asymmetry of the acquired posture.

According to the present invention, various sensors are placed on a person. The sensors will in particular be selected from among pressure sensors, inclinometers, goniometers, gyroscopes, and magnetometers. A processing unit receives by wireless connection the data from the sensors and processes them to determine the relative positions of various body segments as well as their possible displacement.

In particular, pressure sensors are placed on the sole of the foot to determine whether the person is static or dynamic and in what position. Indeed, when a person is standing still, the pressure exerted on the soles of the feet correspond to the person's weight. If a person is sitting still, the pressure exerted on the soles of the feet is much lower than that of his weight, part of the body being supported by the seat. If a person is walking, the pressure varies substantially regularly from a minimum to a maximum, each foot being lifted in turns. The person's general static or dynamic, standing or sitting situation is determined. Sensors enable determining various other data about the person's activity. Other sensors likely to be used are pressure sensors placed on a amputee's stump.

As illustrated in FIGS. 1A, 1B, and 1C, especially when walking, a person is led, in a normally imperceptible natural way, to incline his chest sideways (FIG. 1A), forward (FIG. 1B), and backward (FIG. 1C). The degree, duration, and speed of the chest inclination in each of these directions essentially depend on the speed of the displacement, or even on its acceleration, but also on the person's weight and age. Such chest inclinations are necessary to maintain the postural symmetry.

After an accidental trauma or in the use of a prosthesis, an individual will unconsciously reduce, enhance or suppress some of these inclinations. These modifications are compensated, unconsciously or by rehabilitation, by modifications of the positions of the other body segments to avoid falling. This will thus not affect the individual's balance and stability but causes an imbalance in the postural symmetry. This postural symmetry imbalance translates as an overload on certain body segments, which overload causes a lesion along time.

According to an embodiment of the present invention, kinematic sensors intended to determine the position, speed, and acceleration of the chest are provided. When the combination of these parameters and their repeating reaches a given threshold specific to the person's physical characteristics, a lesion is likely to occur. The processing unit of the device according to an embodiment of the invention compares the data collected by the sensors or a combination of these data with programmable preset thresholds. For example, for a scoliotic person or an older person with lumbar pain, the processing unit compares the angle of the chest with the vertical direction as well as with the lower limbs, compares the angular speed with predefined oscillation areas, and determines the risk of a lesion according to these data.

According to an embodiment of the present invention, when a risk of lesion appears, the processing unit sends a message either to personnel supervising the rehabilitation or, preferably, to the person via any visual, sound, or tactile warning device made available to the person. Communications between the processing unit and the warning device will preferably be performed through a wireless system.

For example, a warning device is a tongue display unit TDU which generally comprises at least one artificial palate intended to be placed in the oral cavity and supporting an array of electrodes likely to enter into contact with at least the upper surface of the tongue. The signal then exhibits a coding capable of activating predefined areas of the electrode array. The coding enables to notify the person either of the existence of a risk of lesion, and possibly the segment(s) causing this risk and, if desired, the degree of the risk of lesion, or an action to be taken to suppress the risk of lesion. The coding mode is selected according to the physical characteristics and to the sensorimotor capacities and to the needs of the affected person, and to his or her capacity to analyze the situation.

It should be noted that the previously-mentioned risk of lesion may be quantified as being a close or distant risk. Thus, a surgeon or a high-level athlete may decide to hold the risky posture, considering that the time for which the posture is held will be too short for a lesion to occur. According to an embodiment, the device, and in particular the processing unit, may take into account the recent history of the postures taken by the bearer to warn him of an increasing risk of lesions. Thus, a surgeon performing a particularly long operation may neglect warnings for long-term risks but will be warned that he is approaching a stage at which one of his body segments will be damaged if he does not modify his posture.

According to another embodiment of the present invention, whether the information on the risk of lesion is transmitted or not to the user, when a lesion is possible, the processing unit triggers the starting of actuators capable of suppressing the risk of lesion.

Such actuators for example are electromyographic actuators or sensory stimulators which cause a stimulation or a vibration of contraction of a tendon, of a tactile receiver, or of a muscle. This stimulation is unconsciously felt by the person as a real movement of his own body and makes him perform an inverse displacement as a reflex. The actuator thus causes a displacement capable of compensating for the risk of lesion.

According to an embodiment of the invention, the processing unit chooses to activate an actuator or to warn the user according to the observed passing. Thus, if the passing of a threshold value is sufficiently low for a reflex motion to be enough to compensate for it, the processing unit will only activate at least one actuator to cause such a compensation. If the passing of the threshold(s) is too large, the central processing unit will warn the user by means of the TDU in one of the previously-described ways.

It should be noted that the thresholds vary significantly from one individual to another. Thus, for a person with lumbar pain or scoliosis, it will be normal for the chest to be significantly inclined with respect to the vertical axis and with respect to the lower limbs.

The device according to the present invention then enables to monitor the inclinations of the trunk/head assembly with respect to the vertical axis, on the one hand, and with respect to the lower limbs on the other hand. According to the extent of the lumbar pain or scoliotic pathology, thresholds are set so that the chest inclination remains within a range which is compatible with the pathology and with the absence of lesions on the lower limbs.

A method and a device for preventing lesions such as described previously may be used in various cases. In particular, they may be used in the context of a rehabilitation or during a recovery phase. They may also be used to enable older persons or persons suffering from spatial perception deficiencies to continue living in their home.

Such a method and such a device have many advantages. In particular, they free the person from a strict or regular surveillance and increase his or her autonomy. Correspondingly, they enable to free personnel and equipments for the follow-up of other persons.

The device is particularly inconspicuous due to the use of a non-visible warning device using a wireless communication protocol between the different elements. This improves the integration of the persons in active life.

For example, after a trauma of a joint such as the knee or the ankle, the flexion thereof must be limited. The possible flexion angle is then progressively increased during the rehabilitation. This is currently obtained by quite constraining systems of immobilization and controlled flexion under control of attendants. It is now possible according to the present invention to fit the person undergoing a rehabilitation with sensors capable of determining the inclination of segments located on either side of the joint and to warn him or her by means of a warning device when he is leaving the authorized flexion area. It is also possible to use the warning device to indicate to a person undergoing a rehabilitation that he or she is stopping a flexion too early and must continue it to reach a threshold value. During the rehabilitation, the threshold value is progressively modified to increase the joint flexion range along time.

Another application is the rehabilitation after an intervention in traumatology, orthopedic surgery, or rheumatology, as well as after the fitting of a prosthesis, especially of the lower limbs. Indeed, after the fitting of a prosthesis, the inclination angle of the rest of the body with respect to the prosthesis and the weight to be supported by the prosthetic limb are limited. The limits become less constraining along time. A conventional problem then encountered is that a person fitted with a prosthesis respects rehabilitation thresholds during sessions, but no longer in between sessions. Such a lack of compliance translates as the application of an excess weight and/or flexion on the prosthetic limb in the first times after the prosthesis has been fit. Another lack of compliance is, on the contrary, an under-utilization of the prosthetic limb. In this last instance, the non-prosthetic limb is submitted to too much stress, which may result in lesions. According to the present invention, it is possible, by means of appropriate sensors, of a processing unit, and of a warning device, to help the person wearing the prosthesis to respect rehabilitation thresholds between specific sessions.

The correction of a postural symmetry to avoid lesions is not limited to a post-traumatic rehabilitation context. The present invention also applies to the prevention of lesions in a professional or leisure context without any traumatic history. Indeed, the combination of sensors, of a processing unit, and of a warning device as well as of possible actuators, may be used to optimize a posture associated with a regular activity. It is thus possible to determine, according to an activity and to the user's characteristics, an optimal position in which the activity is performed with no lesion. For example, for shooters, it is possible to determine an ideal position of the different body segments with respect to one another, to provide shooters with sensors especially intended to determine the angles between the different segments as well as with the weapon, to compare the data with optimal ranges, and to transmit postural symmetry correction data to the shooter via a warning device.

The present invention may also be used preventively in situations other than sports applications. Thus, musculoskeletal disorders linked to a bad working position, for example, in the practice of static tasks such as a musical performance, the typing of texts, and or the entry of data by means of a computer keyboard, are at the origin of various syndromes and lesions among which sore feet, swollen legs, varicose veins, general muscular fatigue, low back pain, stiff neck and/or shoulder joints, . . . Similarly, the seated position in which these activities are performed may cause lesions of the vertebral column. The postural symmetry correction according to the present invention may be used during the training to correct an incorrect position better than a human observer would, and before lesions appear. Further, the device according to the invention being formed of non-bulky and non-stigmatizing elements, it may be used in convenient non-discriminating fashion during the execution of an activity.

The present invention also applies to the control of the seated position of a hemiplegic person. Indeed, a hemiplegic person has significant sensory impairments on the damaged side. Hemiplegia goes along with a decrease in strength generation and maintaining capacities and with an increase in muscle latency times. Current rehabilitations enable a person to compensate for deficiencies and to learn to stand, to move without falling, and to perform various activities, including professional. However, up to now, the occurrence of multiple lesions could be observed on hemiplegic persons. The device of the present invention enables to avoid such lesions either by means of a reflex stimulation or by indicating to the bearer, at any time or in case of risks, that he must modify his posture.

Generally, the method and the device according to the present invention may be used to correct the position of any seated person, especially a disabled person to avoid the occurrence of lesions.

Of course, the present invention is likely to have different variations and modifications which will occur to those skilled in the art. In particular, the number and the nature of the sensors as well as of the possible actuators will be selected according to the targeted application. The sensors will be limited to the considered activity and to the sole segment(s) to be rehabilitated or having a position which must be corrected.

1. A method of rehabilitation or of correction of the postural symmetry of a person in static or dynamic situations, comprising the steps of:
monitoring at least one parameter of at least one body segment of the person;
detecting whether the at least one parameter has passed a low or a high threshold value; and
when the at least one parameter has passed the low or high threshold value, causing said processing unit to warn warning the person by means of an electrical tongue stimulation signal that at least one of the low or the high threshold values has been passed.

2. The method of claim 1, wherein a coding of the electrical tongue stimulation signal is performed according to the location of the body segment where the one of the low or the high threshold value is passed.

3. The method of claim 2, wherein the coding of the electrical tongue stimulation signal is performed according to the extent to which the one of the low or the high threshold value is passed.

4. The method of claim 1, wherein a coding of the electrical tongue stimulation signal is performed to cause the displacement of at least one body segment of the person to compensate for the passing of the low or the high threshold values.

5. The method of claim 1, further comprising a step of, after the detection of a passing, triggering of at least one actuator so as to cause a movement of at least one body segment of the person to compensate for the passing of the low or the high threshold values.

6. The method of claim 5, wherein the actuator is an electromyographic actuator or a sensory stimulator capable of applying a tactile, tendinous, or muscular vibration.

7. A device of rehabilitation or correction of the postural symmetry of a person in static or dynamic situations, comprising:
at least one sensor placed on at least one body segment of the person; and
a processing unit adapted to detect whether a parameter provided by the at least one sensor has passed at least one given threshold and to send a signal representative of the passing to at least one actuator.

8. The device of claim 7, wherein the at least one actuator is a device of electrical stimulation of the tongue.

9. The device of claim 7, wherein the at least one actuator is an electromyographic actuator or a sensory stimulator adapted to cause a postural reaction.
